(19) United States
(12) Patent Application Publication
(10) Pub. No.: US 2011/0217254 A1
(43) Pub. Date: Sep. 8, 2011

(54) ANTIPERSPIRANT PREPARATIONS COMPRISING HYDROTALCITE

(75) Inventors: Heike Miertsch, Hamburg (DE); Stefan Biel, Hamburg (DE)

(73) Assignee: BEIERSDORF AG, Hamburg (DE)

(21) Appl. No.: 13/125,316

(22) PCT Filed: Sep. 23, 2009
(86) PCT No.: PCT/EP09/06866

§ 371 Date: May 20, 2011

(30) Foreign Application Priority Data
Oct. 22, 2008 (DE) 102008052746.7

(51) Int. Cl. A61K 8/28; A61K 8/26; A61Q 15/00

(52) U.S. Cl. 424/66; 424/68

(57) ABSTRACT

Hydrotalcite helps prevent changes in the scent and color of an antiperspirant preparation comprising one or more antiperspirant and/or deodorant active ingredients and a maximum of 5% by weight of water, based on the total mass of the preparation.

The invention comprises antiperspirant preparations having hydrotalcite

Humans have two different forms of sweat glands. The eccrine sweat glands excrete mainly salt and water and usually do not contribute to the development of odor. The sweat glands responsible for the odor are apocrine sweat glands, which excrete fatty acids, cholesterol and other compounds. These substances are decomposed by bacteria on the skin, wherein the degradation products produce the odor typical of sweat.

In order to suppress the odor of sweat over a longer period the use of cosmetic preparations is indispensable. Conventional cosmetic deodorants are based on different principles of action, which can also be combined: on the one hand deodorant active substances are used, which suppress the growth of the bacteria that cause the sweat odor. These germ-inhibiting (bacteriostatic) agents include, for example triclosan, chlorhexidine or naturally occurring compounds such as farnesol and phenoxyethanol.

On the other hand, antiperspirants which prevent the production of sweat by blocking the openings of sweat glands are employed. In by far the majority of antiperspirants the formation of sweat can be reduced by means of astringents—primarily aluminum salts such as aluminum hydroxychloride (aluchlorohydrate) or aluminum/zirconium salts.

The combination of astringents with antimicrobially effective substances in one and the same composition is also customary. Furthermore, perfume substances are used to mask the odor of sweat.

It is disadvantageous in the use of aluminum chlorohydrate, for example, that residue can discolor clothing unpleasantly and the low pH value (acid) of the cosmetic preparation has a disadvantageous effect on the biological balance of the skin.

In addition to liquid deodorants, for example, aerosols, atomizers and roll-ons, solid formulations, for example, deodorant sticks, powder, powder sprays, intimate cleansing agents and the like, are also known and customary.

With antiperspirants a distinction is made between aqueous and anhydrous formulations. The aqueous products are generally emulsions, but also hydroalcoholic solutions or so-called soap gel sticks can be found on the market.

The anhydrous products are usually suspensions, that is, the antiperspirant active ingredient is suspended in powder form in a carrier oil. These anhydrous suspensions are used in liquid form in aerosols, but they can also be formulated with thickeners to form pasty to solid products. Since the release of the antiperspirant active ingredient from anhydrous formulas is not as good as with aqueous systems, the concentration of the antiperspirant active ingredients is also higher. This leads to an increased influence on the stability of the formulations, in particular on the perfume stability. Compared to aqueous products, the perfume impression with anhydrous suspensions can be less fresh. The formulation constituents must therefore be selected such that as little interaction as possible can take place with the antiperspirant salts.

In US 20060127336 and JP 03-190811 pure deodorant preparations are described. In US 20060127336 the preparations comprise aluminosilicates. Hydrotalcite, inter alia, can be added to the preparations as adsorbents. Antiperspirant active ingredients and the disadvantages thereof are not mentioned therein.

DE 102007059678 describes anhydrous antiperspirant sticks, containing at least one lipid component or wax component with a melting point >30° C., at least one oil that is liquid under normal conditions, selected from linear polydimethylsiloxanes having 2 to 50 siloxane units, a maximum of 5% by weight of water, based on the total composition, and at least one antiperspirant active ingredient.

The linear polydimethylsiloxane having 2 to 50 siloxane units is contained in the antiperspirant stick in a total quantity of 10-60% by weight, particularly preferably 20-50% by weight.

U.S. Pat. No. 6,024,945 describes the use of hydrotalcite (dialuminum hexamagnesium carbonate hexadecahydroxide tetrahydrate) in aerosols that contain 1,1-difluoroethane. Hydrotalcite prevents the reaction between aluminum chlorohydrates (ACH) and the difluoroethane, which leads to toxic acetaldehydes and to corrosion of the aerosol can.

EP 1284128 discloses antiperspirant sticks comprising an organic or inorganic material for eliminating the yellowing of the sticks.

EP 1515691 describes the use of aluminum-zirconium glycine chlorohydrates with a low metal-chloride ratio and a stabilizing basic raw material for improving stability and scent. The base can, inter alia, be inorganic, insoluble in water, e.g., magnesium oxide, calcium oxide. The formulation is anhydrous.

DE 102004014294 A1 describes antiperspirant preparations comprising, in addition to mixed oxides, also 2-alkyl-branched acids and/or derivatives thereof, wherein the 2-alkyl-branched acid has 4 to 38 carbon atoms. As mixed oxides, hydrotalcites can, inter alia, be selected. However, the problem of the yellowing of AT preparations is not mentioned.

It is desirable to provide an antiperspirant preparation that does not have the described disadvantages of the prior art. In particular it would be desirable if an AT preparation reduced or prevented the formation of white residues and at the same time has a pleasant feeling on the skin as well as a high perfume stability.

The invention comprises an antiperspirant preparation comprising one or more antiperspirant active ingredients as well as hydrotalcite (dialuminum hexamagnesium carbonate hexadecahydroxide tetrahydrate), wherein the water content is less than 5% by weight, based on the total mass of the preparation. The preparation according to the invention does not comprise any 2-alkyl-branched acids and/or derivatives thereof, if the 2-alkyl-branched acids comprise 4 to 38 carbon atoms.

In order for the hydrotalcite to be able to act as an acid scavenger in the formulation, the addition of water is necessary. However, the addition of water is limited to a maximum of 5% by weight, based on the total mass of the stick.

To activate the hydrotalcite, water is advantageously to be used, in a ratio of water to hydrotalcite of 10:1 to 1:10.

Astringents are used as antiperspirant active ingredients, primarily aluminum compounds. The previously used salts aluminum sulfate or aluminum chloride, which have a very acid action, and agaric acid have largely been replaced by aluminum hydroxychloride and aluminum alcoholates. The following list of antiperspirant active agents to be used advantageously is in no way intended to be limiting:

Aluminum Salts:
Aluminum salts such as aluminum chloride AlCl₃, aluminum sulfate Al₂(SO₄)₃
Aluminum chlorides of the empirical summation formula [Al₂(OH)ₘClₙ], where m+n=6
Aluminum chlorohydrate [Al₂(OH)₅Cl]×H₂O
   Standard Al Complexes: Locron P (Clariant), Micro-Dry (Reheis), ACH-331 (Summit), Aloxicoll PF 40 (Giulini).
   Activated Al Complexes: Reach 501 (Reheis), AACH-324 (Summit), AACH-7171 (Summit), Aloxicoll P (Giulini), Aloxicoll SD100
Aluminum sesquichlorohydrate [Al₂(OH)_{4.5}Cl_{1.5}]×H₂O
   Standard Al Complexes: Aluminum sesquichlorohydrate (Reheis), AACH-308 (Summit)
   Activated Al complexes: Reach 301 (Reheis)
Aluminum dichlorohydrate [Al₂(OH)₄Cl₂]×H₂O

Aluminum Zirconium Salts:
Aluminum/zirconium trichlorohydrex glycine [Al4Zr(OH)13Cl3]×xH2O×Gly
   Standard Al/Zr Complexes: Rezal 33GP (Reheis), AZG-7164 (Summit), Zirkonal P3G (Giulini)
   Activated Al/Zr Complexes: Reach AZZ 902 (Reheis), AAZG-7160 (Summit), Zirkonal AP3G (Giulini)
Aluminum/zirconium tetrachlorohydrex glycine [Al4Zr(OH)12Cl4]×H2O×Gly
   Standard Al/Zr Complexes: Rezal 36G (Reheis), AZG-368 (Summit), Zirkonal L435G (Giulini)
   Activated Al/Zr Complexes: Reach 908 (Reheis), AAZG-7167 (Summit), Zirkonal AP4G (Giulini)
Aluminum/zirconium pentachlorohydrex glycine [Al8Zr(OH)23Cl5]×H2O×Gly
Aluminum/zirconium octachlorohydrex glycine [Al8Zr(OH)20Cl8]×H2O×Gly

However, glycine-free aluminum/zirconium salts can also be advantageous.

The use of antiperspirant salt suspensions or gels can also be advantageous, in which aluminum salts present in powder form are offered dispersed in diverse oils.

The antiperspirant active ingredients are used in the formulas according to the invention in a quantity of from 1 to 35% by weight, preferably from 1 to 25% by weight.

Aluminum salts as AP active ingredients lead to a typical acid odor in formulations. Although this odor can be masked by perfumes, in the course of storage it becomes stronger again at times. Furthermore, aluminum salts can lead to a yellowing of the product, which is undesirable above all in pasty and solid products. The origin of these changes in scent and color could be a reaction by the aluminum salts with constituents of the formulation.

These disadvantages can be reduced or avoided according to the invention by the addition of hydrotalcite in combination with the small amounts of water.

Hydrotalcite is an international non-proprietary name for the antacid effective dialuminum hexamagnesium carbonate hexadecahydroxide tetrahydrate, Al₂O₃.6MgO.CO₂.12H₂O or Al₂Mg₆(OH)₁₆CO₃.4H₂O, Mᵣ 531.92. Hydrotalcite is virtually insoluble in water. However, in order for the hydrotalcite to be able to act as an acid scavenger in the formulation having a low water content, the addition of water is necessary. Hydrotalcite was patented by Kyowa in 1970 and is commercially available from Bayer (Talcid®) and as a generic substance.

Hydrotalcite neutralizes excess gastric acid, which has a harmful effect on the gastric mucosa. For example, heartburn is relieved thereby and gastrointestinal ulcers heal more quickly.

According to the invention hydrotalcite is understood to represent aqueous carbonates with foreign anions. An aluminum hydroxide magnesium carbonate is particularly suitable in this respect, which is offered by SPI Pharma under the trade name Alma 3512.

Furthermore, hydrotalcite is understood to represent synthetically produced aluminum magnesium hydroxycarbonate, which is offered by SüdChemie, for example, and is used as a costabilizer in the production of PVC and polyolefins.

According to the invention, the name hydrotalcite covers compounds with the names aluminum hydroxide magnesium carbonate and dialuminum hexamagnesium carbonate hexadecahydroxide tetrahydrate.

Hydrotalcite is reduced by free chloride released from the antiperspirant active ingredients (AP active agents). The antiperspirant formulations then no longer yellow and the unpleasant odor formation is avoided. This leads to a better perfume release, a better perfume stability and to white products. Although hydrotalcite makes the product itself whiter, the white residues are not intensified.

Hydrotalcite thus serves to avoid changes in scent and color in antiperspirant preparations that comprise one or more antiperspirant and/or deodorant active ingredients and a maximum of 5% by weight of water, based on the total mass of the preparation.

The addition of 2-alkyl branched acids and/or the derivatives thereof, wherein the 2-alkyl branched acid has 4 to 38 carbon atoms, is omitted according to the invention, since an additional effectiveness against bacteria, mycota, mold and viruses is not the object of the present invention.

The addition of water is thereby no more than 5% by weight and thus defines the above-mentioned upper limit of the low water content of the antiperspirant sticks according to the invention.

A combination of hydrotalcite and water in a ratio of 10:1 to 1:10 is advantageous.

Further possible and preferred components of the preparation are at least one oil and at least one structure imparting agent. The structure imparting agent can be present in the form of suspending aids for liquid products or also as thickeners (fat or wax) for pasty to solid products.

According to the use according to the invention, the preparations according to the invention can be offered and designed in different application forms. Advantageously, the AP or deodorant preparations are present in the form of aerosols, that is, in aerosol containers, squeeze bottles or preparations sprayable by means of a pump device.

A form of roll-on devices, as solid deodorant sticks and in the form of pasty formulations applicable from normal bottles and containers is also according to the invention. Furthermore, the preparations can be advantageously present as deodorizing tinctures, deodorizing intimate cleansing agents, deodorizing shampoos, deodorizing shower or bath preparations, deodorizing powders or deodorizing powder sprays.

According to the prior art, anhydrous or low water content (<5% by weight) antiperspirant formulations comprise:
1. a carrier oil and/or a nonvolatile oil (emollients or masking oils),
2. an antiperspirant active agent and
3. a structure imparting agent (thickeners and suspending aids)
   which also represent preferred components according to the invention. The powdery AP active agent is dispersed in the carrier oil, which usually has a high volatility.

The nonvolatile oils influence the sensory properties, thus the emollients are responsible for the feeling on the skin and masking oils reduce white residues.

With the structure imparting agent, a distinction can be made between suspending aids and thickeners. The suspending aids delay the sedimentation of the solid particles in the suspension. The thickeners serve to increase the viscosity and to develop the structure, in particular in the case of pasty to solid systems. They are generally divided into high melting and low melting components.

Oils are water-insoluble organic compounds that are liquid at 20° C. and have a relatively low vapor pressure, the common feature of which is not the corresponding chemical constitution, but the similar physical consistency. Oils are soluble in almost all organic solvents, wherein the solubility does not always correspond to that of the fatty acid components. They differ from the fats, which are likewise composed of triglycerides, through their low melting point, which increases with the chain length and decreasing number of double bonds.

Carrier oils are those oils in anhydrous suspensions which are mainly used to disperse the solids. The carrier oil also plays a major role in the feeling on the skin, since it has a very high content in the formulation as a rule.

High-volatility oils are particularly suitable as carrier oils. Carrier oils are for example oils on a hydrocarbon basis, e.g., isoparaffins and completely synthetic oils, e.g., silicone oils such as cyclomethicone.

Volatile supporting material is understood to represent oils that have a substantial vapor pressure at body temperature. According to the prior art these can be silicone oils, in particular cyclomethicone, and hydrocarbons, in particular isoparaffins.

In anhydrous formulations, in addition to the high-volatile carrier oil, at least one nonvolatile oil, also referred to as an emollient, is often used. Emollients are used to improve the feeling on the skin and can make a major contribution to the avoidance of white residues. Emollients that help to minimize residue are characterized by a relatively high refractive index (>1.40) and are also referred to as masking oils.

Nonvolatile oils preferably come from the group of branched and unbranched hydrocarbons, silicone oils, dialkyl ethers, dialkyl carbonates, the group of saturated or unsaturated, branched alcohols, as well as the fatty acid triglycerides, in particular, the synthetic or natural triglycerin esters of saturated and/or unsaturated, branched and/or unbranched alkane carboxylic acids having a chain length of 8 to 24, the group of esters of saturated and/or unsaturated, branched and/or unbranched alkane monocarboxylic or dicarboxylic acids having a chain length of 1 to 44 C atoms and saturated and/or unsaturated, branched and/or unbranched alcohols or diols having a chain length of 1 to 44 C atoms, from the group of esters or diesters of aromatic and/or non-aromatic carboxylic acids and saturated and/or unsaturated, branched and/or unbranched alcohols (monovalent or multivalent) having a chain length of 1 to 30 C atoms, as long as the oil component or the totality of the oil components are liquid at room temperature.

Structure imparting agents can on the one hand increase the viscosity of a formulation and on the other hand they can serve to stabilize certain raw materials like particles in the formulation. A distinction is made between thickeners and suspending aids.

Thickeners represent those substances that melt at a temperature above room temperature (20° C.) and crystallize during solidification such that the viscosity of the formulation is increased and a semi-solid to solid product can be formed. This means fats and waxes in particular. They are preferably insoluble in water or are immiscible with water in the molten state.

Fats are substances of the plant or animal body that are solid or semi-solid at 20° C., which chemically consist essentially of mixed triglycerides of higher fatty acids with an even number of carbon atoms as well as low contents of acyl lipids (e.g., sterol esters) and unsaponifiable matter. Many foreign constituents such as mineral oils, softeners and biocides, which concentrate in the fat due to their lipophilic character, are likewise present in the unsaponifiable matter. Together with the oils, which are chemically similar, but are liquid at 20° C., the fats are included in the group of lipids.

Fats frequently belong to the low-melting thickeners. Low-melting thickeners are those with a melting temperature below 50° C. For antiperspirant formulations, in particular thickeners are preferred which melt in the range between 30° C. and 45° C. They are preferably selected from the group of triglycerides and/or esters.

High-melting thickeners are understood to be components that melt above 50° C. Waxes of all kinds are particularly suitable. Waxes generally have the following properties: kneadable at 20° C., solid to brittle and hard, coarse to microcrystalline, transparent to opaque, but not glassy; melting above 40° C. without decomposition, relatively low-viscous already slightly above the melting point and not stringy, consistency and solubility highly temperature-dependent, polishable under slight pressure. They differ from similar synthetic or natural products (e.g., resins, plastic masses, metal soaps, etc.) mainly in that they generally change into the molten, low-viscous state between 50 and 90° C. Waxes can be of natural origin, chemically modified or of synthetic origin. In the natural waxes a distinction is made between vegetable origin (e.g., carnauba wax, candelilla wax), animal origin (e.g., beeswax) and mineral origin (e.g., microwaxes, ceresine, ozocerite).

Microwaxes or also microcrystalline waxes are higher melting constituents of petroleum. They are composed of a mixture of saturated hydrocarbons and also contain alkyl-substituted cycloparaffins and alkyl-substituted or naphthene-substituted aromatics. In contrast to coarse-crystalline commercial paraffin, microwax has a very fine crystal structure.

In anhydrous antiperspirant formulations, as solidifying fat or wax components, particularly more or less all saturated linear fatty acids and fatty alcohols having 14-22 C atoms, triglycerides of saturated fatty acids having 14-22 C atoms, saturated linear wax esters (fatty acid-fatty alcohol esters) having 28-44 C atoms, waxes of natural origin, microcrystalline waxes or mixtures thereof are suitable.

Preferably according to the invention microcrystalline waxes, such as ceresine waxes, and wax esters from the group of fatty alcohols and fatty acids having 28 to 44 C atoms are selected.

Suitable thickeners are also synthetic waxes, such as the synthetic wax, Fischer-Tropsch wax from Sasol Sasolwax C80.

According to DE 102007059678, in which the presence of volatile silicone oils, in particular cyclomethicone is excluded, the stick preparation according to the invention does not comprise any polydimethylsiloxanes having 2 to 50 siloxane units.

Suspending aids increase the stability of particles in suspensions. In anhydrous antiperspirant formulations, preferably modified phyllosilicates, clay minerals and/or silicic acids are used.

Advantageous modified phyllosilicates for the purposes of the present invention are, for example, modified smectites. Smectites are very fine-grained (usually <2 mm) three-layered clay minerals chiefly occurring as lamellar, moss-like or spherical aggregates in which a central layer of octahedrally coordinated cations is surrounded in a sandwich-like manner by two layers of [(Si,Al)O4] tetrahedrons. Advantageous modified smectites are, e.g., modified montmorillonites. Montmorillonites are described by the approximate chemical formula Al2[(OH)2/Si4O10].n H2O or Al203.4 SiO2.H2O.n H2O and represent clay minerals belonging to the dioctahedral smectites. Furthermore, modified hectorites are particularly advantageous for the purpose of the present invention. Hectorites belong to the smectites and have the approximate chemical formula M+0.3(Mg2.7Li0.3)[Si4O10(OH)2], wherein M+ usually represents Na+.

Furthermore modified bentonites are advantageous for the purposes of the present invention. Bentonites are clays and rocks, which contain smectites, above all montmorillonites, as the main minerals. “Raw” bentonites are either calcium bentonites (referred to as Fuller earths in Great Britain) or sodium bentonites (also: Wyoming bentonites).

Modified phyllosilicates for the purposes of the present invention are phyllosilicates, in particular the phyllosilicate types already mentioned, the organophilicity (also lipophilicity) of which has been increased—e.g., by reaction with quaternary ammonium compounds. Phyllosilicates of this type are also referred to as organophilic phyllosilicates.

According to the invention stearalkonium hectorite, a reaction product of hectorite and stearalkonium chloride (benzyl dimethyl stearyl ammonium chloride) and quarternium-18 hectorite, a reaction product of hectorite and quaternium-18 are particularly preferred. Quaternium-90 bentonite, a reaction product of bentonite and quaternium-90, is also preferred according to the invention.

In the use of clay minerals, a so-called activator can be used in addition. This has the function of delaminating the clay mineral used, which is also referred to as activation. Usually, to this end, small polar molecules, such as propylene glycol carbonate and ethanol are used, which, with mechanical energy input, are pushed between the layers of the clay mineral lamellae and thus render possible the desired process through electrostatic interaction therewith. Furthermore, they form hydrogen bonds to the delaminated clay mineral flakes and through this bridge function guarantee the cohesion of the “house of playing cards” structure formed. Typically, these systems exhibit a very marked thixotropy.

Silicic acids are compounds having the general formula (SiO2)m·n H2O. According to the invention, the pyrogenic silicic acids are very important. Highly dispersed silicic acids, which are produced by flame hydrolysis (type A) are collected under the designation pyrogenic silicic acids. On their virtually pore-free surface they have far fewer OH groups than precipitated silicic acids. Due to their hydrophilic character by virtue of the silanol groups, the synthetic silicic acids are frequently subjected to a chemical post-treatment process, in which the OH groups react, e.g., with organic chlorosilanes. Modified, e.g., hydrophobic surfaces are thereby produced, which substantially expand the technical application properties of these silicic acids. They can be obtained under the trade names Aerosil and Cab-O-Sil with various properties.

The total quantity of one more suspending aids in the formulations according to the invention is advantageously in the range of from 0.05% to 5.0% by weight, preferably from 0.1% to 3.0% by weight, based on the total mass of the sticks.

Further components of the preparation according to the invention can be: surface-active substances (emulsifiers), fillers, deodorant active ingredients, perfume and propellant gas.

Also, in addition to fatty alcohols, further constituents of conventional cosmetics can be included.

In addition and preferred according to the invention, the AP sticks comprise emulsifiers.

Emulsifiers are auxiliaries for producing and stabilizing emulsions, which can also be referred to as surface-active substances or surfactants and as a rule are present as oily to waxy, as well as powdery substances. Emulsifiers reduce the interfacial tension between the two phases and, in addition to the reduction of the interfacial work, also achieve a stabilization of the emulsion formed. They stabilize the emulsion formed by interfacial films as well as by the formation of steric or electric barriers, which prevents the coalescence of the emulsified particles.

In order for compounds to be able to be effective as emulsifiers, they must have a certain molecular structure. The structural characteristic of compounds of this type is their amphiphilic molecular structure. The molecule of a compound of this type has at least one group with affinity to substances of strong polarity (polar group) and at least one group with affinity to nonpolar substances (apolar group).

In anhydrous suspensions the emulsifiers are used to improve the ability of the formulation to be washed off the skin. The structure imparting agents advantageously contained in the anhydrous formula can leave perceptibly waxy residues on the skin. Through the presence of polar groups in the emulsifiers used, when the formulation is being washed off the affinity to water is increased and the residues disappear. Preferably nonionic emulsifiers are suitable for this purpose.

Nonionic emulsifiers mean surface-active substances that do not form ions in aqueous solution. The hydrophilic character of nonionic emulsifiers of this type is achieved through the content of the polar groups in the molecule. The nonionic emulsifiers include fatty alcohols (e.g., cetyl alcohol or stearyl alcohol), partial fatty acid esters of polyvalent alcohols with saturated fatty acids (e.g., glycerol monostearate), partial fatty acid esters of polyvalent alcohols with unsaturated fatty acids (e.g., glycerol monooleate, pentaerythritol monooleate), furthermore polyoxyethylene esters of fatty acids (e.g., polyoxyethylene stearate), polymerization products of ethylene oxide and propylene oxide on fatty alcohols (fatty alcohol polyglycol ether) or fatty acids (fatty acid ethoxylates).

The emulsifiers are used in the formulations according to the invention in a quantity of 0.1% to 10% by weight, preferably from 0.2% by weight to 5% by weight.

Furthermore, the sticks according to the invention can also contain fillers.

Fillers are particulate raw materials, which behave inertly with respect to the other formulation constituents. They essentially are important for the feeling on the skin, but can also influence the appearance and structure of the formulation. Simple, neutral fillers are preferably talc and kaolin, but polysaccharides, such as starches and cellulose and the derivatives thereof are also suitable as fillers.

The fillers are used in the formulations according to the invention in a quantity of 0.5 to 25% by weight, preferably from 1% by weight to 20% by weight.

Advantageously, the preparations according to the invention are formulated as solid preparations, for example, as deodorant sticks.

The following examples are designed to clarify the present invention without limiting it. Unless stated otherwise, all quantities, proportions and percentages are based on the weight and the total quantity or on the total weight of the preparations. The weight percentages given in the examples are active contents.

### EXAMPLES

| Sticks | Example 1 | Example 2 | Example 3 |
|---|---|---|---|
| Hydrogenated castor oil | 1.5 | | 1.5 |
| Stearyl alcohol | 20 | 5 | 20 |
| Glyceryl stearate SE | 0.5 | | |
| Sasolwax C80¹ | | 2 | |
| Ceresine Isco Care 126² | | 10 | |
| Cetyl palmitate | | 7.5 | |
| PPG-14 Butyl ether | 15 | | 15 |
| Cyclomethicone | 41 | 28.5 | 40 |
| Caprylic/Capric triglyceride | | 25 | |
| Water | 0.5 | 0.5 | 1.5 |
| Aluminum hydroxide | 0.5 | 0.5 | 1 |
| Magnesium carbonate³ | | | |
| Aluminum zirconium | 16 | 16 | 16 |
| Tetrachlorohydrex GLY | | | |
| Talc | 4 | 4 | 4 |
| Perfume | 1 | 1 | 1 |
| Aerosols | Example 4 | Example 5 | Example 6 |
| Cyclomethicone | 7.550 | 6.125 | 11.125 |
| Dimethicone | 0.450 | | 1.000 |
| C12-15 Alkylbenzoate | | 2.250 | |
| Disteardimonium hectorite | 0.600 | 0.525 | 0.700 |
| Water | 0.075 | 0.050 | 0.100 |
| Aluminum hydroxide | 0.075 | 0.050 | 0.075 |
| Magnesium carbonate³ | | | |
| Aluminum chlorohydrate | 5.250 | 5.000 | 6.000 |
| Perfume | 1.000 | 1.000 | 1.000 |
| Propellant gas (mixture of propane, | 85.000 | 85.000 | 80.000 |
| butane and isobutane) | | | |

¹Synthetic wax, Fischer-Tropsch wax from Sasol
²Microcrystalline wax, ceresine wax from PARAMELT BV
³Hydrotalcite, Alma 3512 from SPI PHARMA

1.-5. (canceled)

6. An antiperspirant preparation, wherein the preparation comprises one or more antiperspirant active ingredients, hydrotalcite, and not more than 5% by weight of water, based on a total weight of the preparation, the preparation being free of 2-alkyl-branched carboxylic acids comprising from 4 to 38 carbon atoms and derivatives thereof.

7. The preparation of claim 6, wherein the preparation further comprises at least on substance selected from carrier oils, thickeners, suspending aids and emulsifiers.

8. The preparation of claim 7, wherein the preparation comprises at least one volatile silicone oil as carrier oil.

9. The preparation of claim 7, wherein the preparation comprises at least one microcrystalline wax as thickener.

10. The preparation of claim 7, wherein the preparation comprises at least one substance selected from smectites, hectorites, bentonites, and stearalkonium hectorite as suspending aid.

11. The preparation of claim 7, wherein the preparation comprises at least one of cetyl alcohol and stearyl alcohol as emulsifier.

12. The preparation of claim 6, wherein the preparation is free of linear polydimethylsiloxanes having from 2 to 50 siloxane units.

13. The preparation of claim 6, wherein the preparation comprises a weight ratio of hydrotalcite and water of from 1:10 to 10:1.

14. The preparation of claim 6, wherein the preparation comprises from 1% to 35% by weight of the one or more antiperspirant active ingredients.

15. The preparation of claim 6, wherein the preparation comprises from 1% to 25% by weight of the one or more antiperspirant active ingredients.

16. The preparation of claim 6, wherein the one or more antiperspirant active ingredients comprise at least one of an aluminum salt and an aluminum zirconium salt.

17. The preparation of claim 6, wherein the preparation the one or more antiperspirant active ingredients comprise aluminum chlorohydrate.

18. The preparation of claim 6, wherein the preparation is present as a stick.

19. The preparation of claim 6, wherein the preparation is present as an aerosol.

20. An antiperspirant preparation, wherein the preparation comprises from 1% to 25% by weight, based on a total weight of the preparation, of one or more antiperspirant active ingredients which comprise at least one of an aluminum salt and an aluminum zirconium salt, hydrotalcite, and less than 5% by weight of water, a weight ratio of hydrotalcite and water being from 1:10 to 10:1, the preparation being free of linear polydimethylsiloxanes having from 2 to 50 siloxane units and of 2-alkyl-branched carboxylic acids comprising from 4 to 38 carbon atoms and derivatives thereof.

21. The preparation of claim 20, wherein the preparation further comprises at least on substance selected from carrier oils, thickeners, suspending aids and emulsifiers.

22. The preparation of claim 21, wherein the preparation comprises at least one substance selected from volatile silicone oils, microcrystalline waxes, smectites, hectorites, bentonites, stearalkonium hectorite, cetyl alcohol, and stearyl alcohol.

23. The preparation of claim 20, wherein the preparation is present as a stick.

24. The preparation of claim 20, wherein the preparation is present as an aerosol.

25. A method of reducing or preventing changes in scent and/or color of an antiperspirant preparation that comprises one or more antiperspirant and/or deodorant active ingredients and not more than 5% by weight of water, based on a total weight of the preparation, wherein the method comprises incorporating in the preparation hydrotalcite.
